(19) United States
(12) Patent Application Publication
(10) Pub. No.: US 2024/0016481 A1
(43) Pub. Date: Jan. 18, 2024

(54) System and method to provide improved urine collection device

(71) Applicant: Claudiney Santos Borges, Spencer, MA (US)

(72) Inventor: Claudiney Santos Borges, Spencer, MA (US)

(21) Appl. No.: 17/863,399

(22) Filed: Jul. 13, 2022

(51) Int. Cl. A61B 10/00

(52) U.S. Cl. CPC A61B 10/007

(57) ABSTRACT

A urinal sample device in form of a nurses' hat which allows males or females to accurately aim the urine where the assembly can be placed at toilet rim and not miss the toilet and allows to keep the clothes and toilet rim clean after use. This eliminates wetting oneself and messing the floor around the toilet and remove the feces from urine. Furthermore, the detached assembly can be cleaned and disposed of if required.

### COPYRIGHT NOTICE

A portion of the disclosure of this patent document contains material winch is subject to copyright protection. The copyright owner has no objection to the facsimile reproduction by anyone of the patent disclosure, as it appears in the Patent and Trademark Office patent files or records, hut otherwise reserves all copyright rights whatsoever.

### BACKGROUND

### Field of the Invention

The present disclosure generally relates to devices, systems, and methods for collecting urine discharged from the body of a user and separation the urine from feces with improved structure of urine collection device.

### Description of the Related Art

It is difficult for many people to hold a urine sample cup effectively to catch their own urine stream flow. It is even more difficult for the elderly, disabled, overweight, pregnant and very young to give urine samples. It is not easy or sanitary for anyone.

When patients or sample-givers are faced with the indignity of someone else holding the urine sample cup for them, it sometimes becomes impossible for the patient to pass urine on demand. It is also time consuming, unsanitary and difficult for the nurses.

A “nurse's hat” is a large plastic disposable pot that looks like an upside-down hat in which the brim is the funnel and the bowl collects the urine flow. After the urine sample is deposited into the nurse's hat, the nurse pours a portion of the urine into a sample cup. The typical nurse's hat entails the placing of it in the toilet. It occupies half of the toilet, so when the patient sits on the toilet it catches the urine. Many times the patient sits back and the urine goes down the toilet unmatched. Another issue with nurse's hat is that it can collect feces in addition to urine which makes it difficult to have an accurate measure of the proper urine amount. Furthermore, this is time consuming and unsanitary for the nurses and patients.

By looking at prior art multiple advancements have been seen in urine collection devices. For instance, a U.S. Pat. No. 7,871,385B2 discloses Urine sample collection device. The urine sample collection device comprising a urine receptor having an outlet aperture, a generally elongate tubular member extending from said receptor outlet aperture to an open end, the tubular member having an opening (21) formed in the side thereof, a coupling means (5′) for releasably mounting a urine collection container, the coupling means having a passage extending therethrough which meets said opening whereby urine can flow from the tubular member into a mounted container, and a flow director (24) located at or adjacent said opening and formed to direct urine past the opening.

A U.S. Pat. No. 5,069,878A discusses Integrity preserving and determining urine sample collection apparatus. A drug testing urine sample collector includes a multiple tamper-proof system to make sure that freshly voided samples of urine are not replaced or adulterated. A chemical type thermometer is located in the reservoir to make sure that the urine sample is fresh when collected. A specific gravity sensitive ball valve restricts entry of a diluted urine sample into the reservoir. The cap for the reservoir also acts as a base for the collector and includes sample adulteration detection reagents. The collector structure prevents the sample from being emptied from the reservoir.

A U.S. Pat. No. 6,740,066B2 discusses Urine collection device. The device is for collecting urine includes a container kept at vacuum, an interface locatable at the urethral opening of a person and a pump operable to create a vacuum within the container. A sensor senses the presence of urine at the interface and opens a valve to allow the pumping of urine from the interface to the container.

A U.S. Pat. No. 10,857,025B2 on Devices and methods for urine collection discloses a device for collecting urine discharged from a body of a user includes a fluid collection assembly having at least one layer for drawing urine discharged from the body into an interior cavity, an external covering that covers a portion of the at least one layer, and at least one fenestration for receiving urine, wherein the fenestration is a portion of the fluid collection assembly that is uncovered by the external covering. The device further includes a cap enclosing a first end of the assembly, a tube having a first end in fluid communication with the cap, and a shape retaining element configured to conform the assembly to a curved configuration and maintain the curved configuration until the configuration is adjusted. The assembly is configured to be disposed against the body of the user, with the at least one fenestration in operative relation with a urethral opening of the user.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable.

It has to be noted that the current invention proposes an assembly with an advancement where the design provides ease of use and enhanced options to both males and females. The proposed design isn't limited to a single gender and is universal in nature, thus facilitating every individual who need the device for health and hygienic reasons. The design of proposed assembly is a hat shaped structure which covers the toilet seat properly thus preventing the spills and uncomfortable seating. It also has removable filter allowing the urine to collect in the hollow cavity leaving feces at the top.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a urine sample collection nurse's hat for males and females with enhanced functionalities.

Another object of the present invention is to provide a urine sample collection nurse's hat with improved structure able to cover toilet rim completely to prevent any loss of urine.

Another object of this invention is to provide a new and improved assembly which provides a removable filter top to drain the urine into collection place keeping the feces at upper removeable filter top.

Yet another object of the present invention is to provide a structure that is relatively inexpensive to produce so that ownership is available to a substantial majority of families.

It is also the objective of the invention to provide a smart assembly which provide ease of use and convenience to everyone.

It is a further aspect of the present invention to provide a new and improved assembly which is of a durable and reliable construction and can sustain its effectiveness after multiple and consistent use.

Thus, it is the objective to provide a new and improved urine sample collection device. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows complete view of old assembly as per preferred embodiments of the invention.

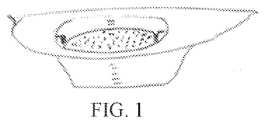

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The device as per its preferred embodiments is a urinal collection sample device designed to help patients/people to collect their urine sample while avoiding wetting/soiling their garment and to avoid bacterial infection.

The urine collection device as per its further embodiments works by the patient/person sitting over the toilet bowl in a squatting position; while the collection device is placed on the toilet bowl. Once comfortably in place the person can then urinate freely without the worry of losing the urine, wetting/soiling their garment/clothing; and avoid bacterial contamination.

The proposed assembly is made of plastic; and is used by people easily with same effectiveness over longer period of time.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. An enhanced nurse's hat structure to facilitate directional urination and separation of urine from feces, comprising:
a support apparatus for supporting the urine collection device on a toilet;
a collection section for receiving and directing urine into the sample holder; and
a flow divider located in the collection section that separates feces from the urine sample;
wherein the flow divider includes a cavity in the collection section that collects the first portion of the urine and a hole at a bottom end of the cavity that directs the first portion of the urine into the toilet.
